(19) United States
(12) Patent Application Publication
(10) Pub. No.: US 2026/0148865 A1
(43) Pub. Date: May 28, 2026

(54) SYSTEM AND METHOD FOR PREDICTING EFFICACY OF COMBINATION ANTICANCER DRUG

(71) Applicant: GWANGJU INSTITUTE OF SCIENCE AND TECHNOLOGY, Gwangju (KR)

(72) Inventors: Hojung NAM, Gwangju (KR); Iljung JIN, Gwangju (KR)

(73) Assignee: GWANGJU INSTITUTE OF SCIENCE AND TECHNOLOGY, Gwangju (KR)

(21) Appl. No.: 19/358,649

(22) Filed: Oct. 15, 2025

(30) Foreign Application Priority Data
Nov. 22, 2024 (KR) 10-2024-0168438

(51) Int. Cl. G16H 70/40

(52) U.S. Cl. CPC G16H 70/40

(57) ABSTRACT

Disclosed herein is a system for predicting efficacy of a combination anticancer drug. A system for predicting efficacy of a combination anticancer drug of the present invention includes: a processor configured to compute efficacy of a combination anticancer drug based on first drug information and second drug information using a neural network model; and a memory, wherein the memory comprises: a first neural network model configured to generate first efficacy-related information from the first drug information and target cancer cell information, and to generate second efficacy-related information from the second drug information and the target cancer cell information; and a second neural network model configured to generate combination efficacy information from the first efficacy-related information, the second efficacy-related information, and the target cancer cell information.

### CROSS REFERENCE TO RELATED APPLICATION

The present application claims priority to Korean Patent Application No. 10-2024-0168438, filed on Nov. 22, 2024, the entire contents of which are hereby incorporated by reference in its entirety.

### BACKGROUND OF THE INVENTION

### Field of the Invention

The present invention relates to a system and method for predicting efficacy of a combination anticancer drug.

### Description of the Related Art

Compared to a single anticancer drug treatment using a single anticancer drug, a combination anticancer drug treatment using a plurality of anticancer drugs may achieve efficacy even at a relatively low concentration compared to a single anticancer drug, may have a synergistic effect achieving a higher effect than each anticancer drug efficacy, and has an advantage of being able to avoid drug-induced immunity.

However, only a very small number of combination anticancer drugs have a synergistic effect, and in most cases, the damage due to side effects may be large, so careful attention is required in exploring effective combination anticancer drug combinations.

In the related art, combination anticancer drug efficacy prediction predicted a synergy score regardless of drug concentration, which could be a potential problem. The synergy score is a relative index that evaluates how much better a combination anticancer drug is compared to a single anticancer drug. However, the efficacy and synergistic effect of a combination anticancer drug highly depend on the concentration of the drug, and are greatly influenced by the efficacy of a single anticancer drug, so it is necessary to consider these in combination.

### SUMMARY OF THE INVENTION

The technical object to be solved by the present invention is to provide a system and method for predicting efficacy of a combination anticancer drug, which is capable of predicting the efficacy considering the synergistic effect according to the concentrations of the combination anticancer drug.

To solve the technical object described above, there is provided a system for predicting efficacy of a combination anticancer drug, according to an embodiment of the present invention. The system may include: a processor configured to compute efficacy of a combination anticancer drug based on first drug information and second drug information using a neural network model; and a memory, in which the memory may include: a first neural network model configured to generate first efficacy-related information from the first drug information and target cancer cell information, and to generate second efficacy-related information from the second drug information and the target cancer cell information; and

a second neural network model configured to generate combination efficacy information from the first efficacy-related information, the second efficacy-related information, and the target cancer cell information, and the first efficacy-related information and the second efficacy-related information may be respectively generated based on a first concentration of the first drug and a second concentration of the second drug.

In an embodiment of the present invention, the first efficacy-related information may include first pathway attention information, and the second efficacy-related information may include second pathway attention information.

In an embodiment of the present invention, the second neural network model may include at least one deep-learning neural network (DNN) for computing a first efficacy ratio in which the first drug contributes to the combination efficacy, and a second efficacy ratio in which the second drug contributes to the combination efficacy.

In an embodiment of the present invention, the second neural network model may further include a synergy computation network for computing synergistic efficacy in which a synergistic effect of the first drug and the second drug contributes to the combination efficacy.

In an embodiment of the present invention, the first neural network model may be pre-trained with a first dataset determined from a first database which include more response information of a target cancer cell to a single drug than a second database, and then fine-tuned with a second dataset including single drug information, target cancer cell information, and single drug response information of the second database, in which response information of the target cancer cell to both the single drug and a combination drug formed by combining the single drug is present.

In an embodiment of the present invention, the second neural network model may be trained with a third dataset including combination drug information, target cancer cell information, and combination drug response information from the second database.

In addition, to solve the technical object described above, there is provided a method for predicting efficacy of a combination anticancer drug, according to an embodiment of the present invention, executed by at least one processor of a computing device. The method may include: determining first drug information, second drug information, and target cancer cell information; generating first efficacy-related information from the first drug information and the target cancer cell information; generating second efficacy-related information from the second drug information and the target cancer cell information; and generating combination efficacy information from the first efficacy-related information, the second efficacy-related information, and the target cancer cell information, in which the first efficacy-related information and the second efficacy-related information may be respectively generated based on a first concentration of the first drug and a second concentration of the second drug.

The present invention has an effect in that it may predict the efficacy considering the synergistic effect according to the concentrations of a combination anticancer drug, and thus not only enables prediction of the effect according to the combination, but also allows proposal of appropriate concentrations.

### BRIEF DESCRIPTION OF THE DRAWINGS

FIG. 1 illustrates a system for predicting efficacy of a combination anticancer drug according to an embodiment of the present invention.

FIG. 2 illustrates in detail a partial configuration of the system for predicting efficacy of a combination anticancer drug according to an embodiment of the present invention.

FIG. 3 illustrates in detail a first neural network model, which is a partial configuration of the system for predicting efficacy of a combination anticancer drug according to an embodiment of the present invention.

FIG. 4 schematically illustrates a detailed structure and operation of the first neural network model.

FIG. 5 illustrates in detail a second neural network model, which is a partial configuration of the system for predicting efficacy of a combination anticancer drug according to an embodiment of the present invention.

FIG. 6 schematically illustrates a detailed structure and operation of the second neural network model.

FIG. 7 illustrates a method for predicting efficacy of a combination anticancer drug according to an embodiment of the present invention.

FIG. 8 illustrates in detail a partial configuration of the method for predicting efficacy of a combination anticancer drug according to an embodiment of the present invention.

FIG. 9 illustrates in detail a partial configuration of the method for predicting efficacy of a combination anticancer drug according to an embodiment of the present invention.

FIG. 10 illustrates in detail a partial configuration of the method for predicting efficacy of a combination anticancer drug according to an embodiment of the present invention.

FIG. 11 illustrates the performance of a pre-trained first neural network model.

FIG. 12 illustrates the performance of a fine-tuned first neural network model.

FIG. 13 illustrates the performance of the second neural network model.

FIG. 14 illustrates the performance of the system for predicting efficacy of a combination anticancer drug depending on cancer type.

FIG. 15 illustrates viability prediction depending on the concentration of component drugs of the combination anticancer drug.

FIG. 16 illustrates a synergistic effect depending on the concentration of component drugs of the combination anticancer drug.

FIG. 17 is a block diagram illustrating an embodiment of a computing system in which the present invention can be implemented.

FIGS. 18 and 19 are block diagrams illustrating an embodiment of a computing device according to the present invention.

### DETAILED DESCRIPTION OF THE INVENTION

The present invention may be variously modified and may have various embodiments, and particular embodiments illustrated in the drawings will be described in detail below. However, the description of the exemplary embodiments is not intended to limit the present invention to the particular exemplary embodiments, but it should be understood that the present invention is to cover all modifications, equivalents and alternatives falling within the spirit and technical scope of the present invention.

In the description of the present invention, the specific descriptions of publicly known related technologies will be omitted when it is determined that the specific descriptions may obscure the subject matter of the present invention.

Hereinafter, the embodiments of the present invention will be described in detail with reference to the accompanying drawings.

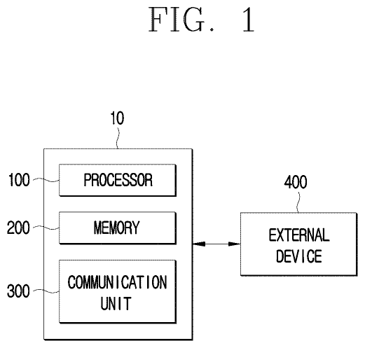

FIG. 1 illustrates a system 10 for predicting efficacy of a combination anticancer drug according to an embodiment of the present invention.

With reference to FIG. 1, a system 10 for predicting efficacy of a combination anticancer drug according to an embodiment of the present invention includes a processor 100, a memory 200, and a communication unit 300.

The processor 100 is connected to the memory 200 and the communication unit 300, and collects information and controls them.

The processor 100 may be configured as a single physical entity, but may also be configured as a plurality of entities. The processor 100 configured with a plurality of entities may process by dividing a single execution element or may process by separating a plurality of execution elements.

The processor 100 may include at least one of a central processing unit (CPU), a graphic processing unit (GPU), a microprocessor, or an artificial intelligence dedicated processor, and the type of the processor is not limited thereto as long as it performs the functions of the present invention.

The memory 200 may store a program, which is a set of data and executable instructions that may be read or written by the processor 100. In particular, the memory 200 may store an artificial intelligence neural network model, a network constituting the model, a module, or the like.

The memory 200 includes storage of non-volatile nature, which may retain data (information) regardless of power supply, and memory of volatile nature, into which data is loaded for processing by the processor and which cannot retain data unless power is provided. The storage may include a flash memory, a hard-disc drive (HDD), a solid-state drive (SSD), or a read only memory (ROM), and the memory may include a buffer, a random access memory (RAM), or a cache.

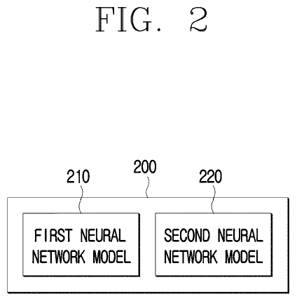

FIG. 2 illustrates in detail the memory 200.

With reference to FIG. 2, the memory 200 includes a first neural network model 210 and a second neural network model 220.

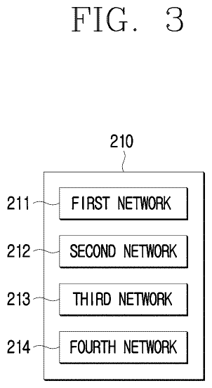

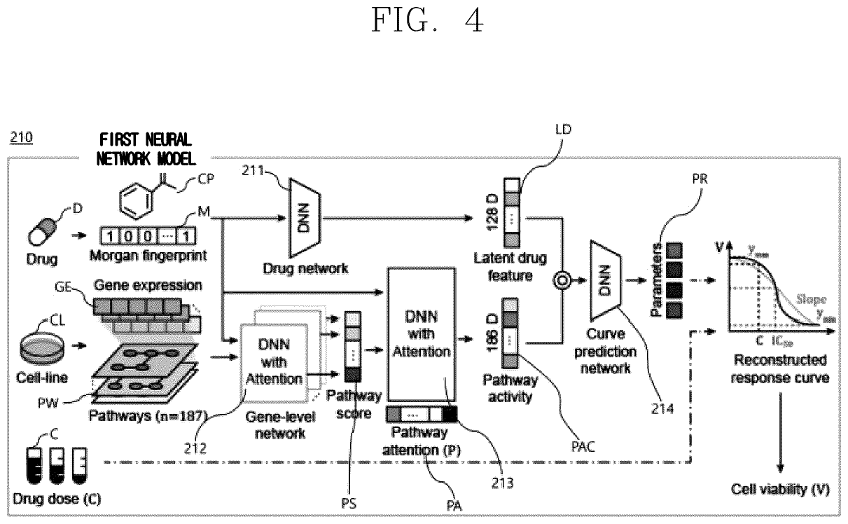

FIG. 3 illustrates in detail the first neural network model 210, and FIG. 4 schematically illustrates a detailed structure and operation of the first neural network model 210.

With reference to FIGS. 3 and 4, the first neural network model 210 includes a first network 211, a second network 212, a third network 213, and a fourth network 214.

The first network 211 outputs a latent drug feature vector LD from drug information.

Specifically, the first network 211 receives information converted into a morgan fingerprint format for a main compound included in a drug as input, and outputs a latent drug feature vector.

The first network 211 is a deep-learning neural network (DNN), and is trained to output features of a latent drug.

The second network 212 outputs a pathway score PS regarding the importance of pathways, with cell line information of a cancer cell and drug information as input.

The cell line information includes gene expression information and information on pathways.

The second network 212 includes a deep-learning neural network (DNN) structure based on attention, and may be referred to as a gene-level network. The second network 212 may learn a correlation with a drug at a gene level.

The third network 213 outputs a pathway activity PAC with the pathway score and drug information as input. When the pathway score PS relates to the importance of a pathway, the pathway activity PAC may be an index representing the activation degree of the pathway by the drug.

In the process of outputting the pathway activity PAC, the third network 213 computes a pathway attention (PA).

The pathway attention PA may be used in the computation of the second neural network model 220.

The third network 213 includes a deep-learning neural network (DNN) structure based on attention and may be referred to as a pathway-level network. The third network 213 may learn a correlation with a drug at a pathway level.

The latent drug feature vector LD is concatenated with the pathway activity PAC and is input to the fourth network 214.

The fourth network 214 may output a single anticancer drug prediction parameter by the concatenated latent drug feature vector LD and pathway activity PAC. The fourth network 214 may output efficacy itself instead of outputting the prediction parameter. In this case, a drug concentration C may be additionally input.

The fourth network 214 includes a deep-learning neural network (DNN) structure, and learns a relationship between drug response and the latent drug feature and pathway activity.

The prediction parameter includes four types of parameters, and a response curve for viability V of a cancer cell according to drug concentration C may be derived from the prediction parameter.

The prediction parameter includes a starting point of the response curve, a viability according to the maximum drug concentration, a concentration corresponding to IC50, and an inclination degree (slope) of the response curve. The concentration corresponding to IC50 refers to a drug concentration that achieves half of the maximum effect of the drug.

Regarding the prediction parameter, when expressed as an equation, it is as follows in [Equation 1].

$$y = {y}_{min} + \frac{{y}_{max} - {y}_{min}}{1 + {e}^{k ( x - IC 50 )}} \qquad [ Equation 1 ]$$

(Here, y is viability, x is drug concentration, yₘᵢₙ is minimum viability, yₘₐₓ is maximum viability, k is the slope of the curve, and IC50 is the aforementioned IC50 concentration.)

The response curve is illustrated on the far right of FIG. 4.

The training of the first neural network model 210 may be performed by a method of pre-training followed by fine-tuning.

A first dataset used for pre-training is based on data provided by the NCI60 database. The first dataset uses 66 cell lines, 50,893 drugs, and 10,105,780 response information from the NCI60 database.

Although the NCI60 data does not include response data on combination anticancer drugs, it is effective as pre-training data for predicting efficacy of single anticancer drugs, as it has a sufficiently large number of drugs and response information.

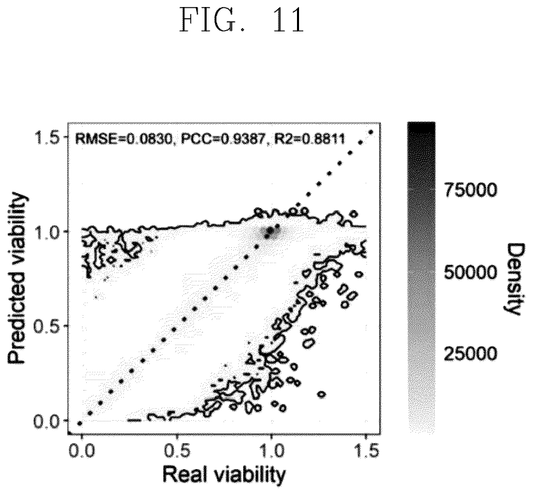

FIG. 11 illustrates the performance of the pre-trained first neural network model 210.

The performance of the first neural network model 210 is visualized as a graph in which the viability of the validation data (Real viability, x-axis), the viability of the predicted data (Predicted viability, y-axis), and the density of data (represented by color intensity) are plotted.

On the top of the graph, the Root Mean Square Error (RMSE), Pearson Correlation Coefficient (PCC), and R-squared (R2) are displayed respectively.

The RMSE, PCC, and R2 of the pre-trained first neural network model 210 are 0.0830, 0.9387, and 0.8811, respectively.

A second dataset used for fine-tuning is based on data provided by the NCI-ALMANAC database. The second dataset includes 44 cell lines, 102 drugs, and 35,041 response information from the NCI-ALMANAC database.

Although the second dataset includes a relatively smaller number of drugs and response information, it is suitable for fine-tuning because it includes response information on drugs associated with information on combination anticancer drugs.

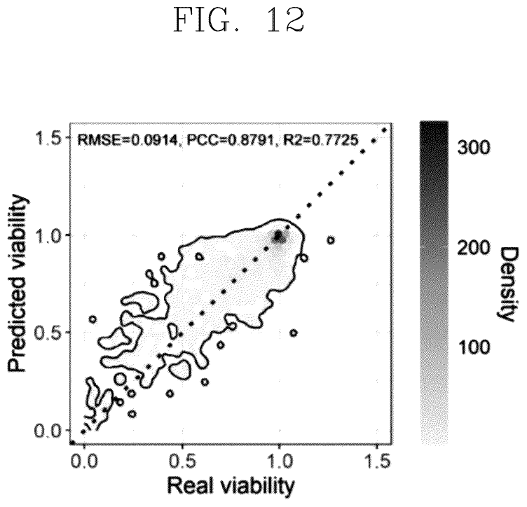

FIG. 12 illustrates the performance of a fine-tuned first neural network model.

The performance of the fine-tuned first neural network model 210 is visualized as a graph in which the viability of the validation data (Real viability, x-axis), the viability of the predicted data (Predicted viability, y-axis), and the density of data (represented by color intensity) are plotted.

On the top of the graph, the Root Mean Square Error (RMSE), Pearson Correlation Coefficient (PCC), and R-squared (R2) are displayed respectively.

The RMSE, PCC, and R2 of the fine-tuned first neural network model 210 are 0.0914, 0.8791, and 0.7725, respectively.

As a prior note, the third dataset used for training of the second neural network model 220 includes 5,032 combination drugs formed by combining 44 cell lines and 102 drugs from the NCI-ALMANAC database, and includes 1,981,135 response information for the combination drugs. The 102 drugs included in the second dataset and the third dataset may be the same.

[Table 1] illustrates the sources and compositions of the first to third datasets.

TABLE 1

| | | | | Number of | |
|---|---|---|---|---|---|
| | | Number of | Number of | combination | Number of |
| Dataset | Database | cell lines | drugs | drugs | responses |
| First dataset | NCI60 | 66 | 50,893 | — | 10,105,780 |
| Second dataset | NCI-ALMANAC | 44 | 102 | — | 35,041 |
| Third dataset | NCI-ALMANAC | 44 | 102 | 5,032 | 1,981,135 |

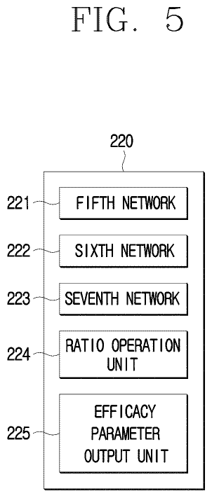

FIG. 5 illustrates in detail the second neural network model 220.

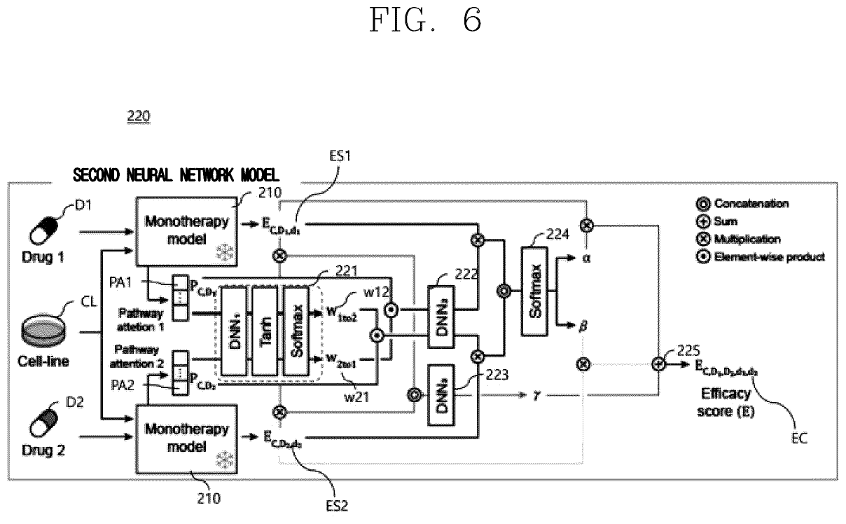

FIG. 6 schematically illustrates a detailed structure and operation of the second neural network model 220.

With reference to FIGS. 5 and 6, the second neural network model 220 is an artificial intelligence neural network model designed to predict combination efficacy, by receiving, as input, a first drug D1 and its concentration d1, a second drug D2 and its concentration d2, and cell line (CL) information.

In addition, the second neural network model 220 operates by partially using operation results of the first neural network model 210. Specifically, the first neural network model 210 outputs first efficacy ES1 and first pathway attention PA1 information from the first drug D1 and cell line CL information. In parallel or sequentially, the first neural network model 210 outputs second efficacy ES2 and second pathway attention PA2 information from the second drug D2 and cell line CL information. The output values of the first neural network model 210 are used as inputs of at least one network of the second neural network model 220.

The second neural network model 220 includes a fifth network 221, a sixth network 222, a seventh network 223, a ratio operation unit 224, and an efficacy parameter output unit 225.

The fifth network 221 may include a deep-learning neural network (DNN) structure, a Tanh activation function, and a Softmax function.

The fifth network 221 outputs a first weight w12 from the first pathway attention PA1. The first weight w12 is element-wise multiplied with the second pathway attention PA2 and input to the sixth network 222.

In parallel or sequentially, the fifth network 221 outputs a second weight w21 from the second pathway attention PA2. The second weight w21 is element-wise multiplied with the first pathway attention PA1 and input to the sixth network 222.

The first weight w12 and the second weight w21 may be expressed by [Equation 2] and [Equation 3], respectively.

$${w}_{1 to 2} = softmax ( Tanh ( {DNN}_{1} ( {P}_{C , {D}_{1}} ) ) ) \qquad [ Equation 2 ]$$

$${w}_{2 to 1} = softmax ( Tanh ( {DNN}_{1} ( {P}_{C , {D}_{2}} ) ) ) \qquad [ Equation 3 ]$$

In [Equation 2] and [Equation 3], DNN₁ denotes a deep-learning neural network included in the fifth network 221, P denotes pathway attention, and a subscript C denotes a cell line, and Dᵢ denotes the i-th drug (compound), respectively. For example, P_{C, D2} indicates pathway attention for which the cell line C and the second drug D₂ are used as input information. In addition, it should be noted that characters expressed in the equations may be different from the reference numerals.

The fifth network 221 may be understood as a module that calculates interaction of a single anticancer drug.

The sixth network 222 includes a deep-learning neural network (DNN) structure. The sixth network 222 may be understood as a module that calculates the influence of a single anticancer drug.

The sixth network 222 generates first output information by using the element-wise multiplied first weight w12 and second pathway attention PA2 as input.

In parallel or sequentially, the sixth network 222 generates second output information by using the element-wise multiplied second weight w21 and first pathway attention PA1 as input.

The first output information is multiplied with the first efficacy information ES1, and the second output information is multiplied with the second efficacy information ES2, and the results of the respective multiplications are concatenated and input to the ratio operation unit 224.

The ratio operation unit 224 includes a Softmax function, and outputs a first efficacy ratio α and a second efficacy ratio β.

The first output information, the second output information, the first efficacy ratio α, and the second efficacy ratio β may be represented by [Equation 4] to [Equation 6] below.

$${α}_{pre} = {E}_{C , {D}_{1} , {d}_{1}} * {DNN}_{2} ( {P}_{C , {D}_{1}} ⊙ {w}_{2 to 1} ) \qquad [ Equation 4 ]$$

$${β}_{pre} = {E}_{C , {D}_{2} , {d}_{2}} * {DNN}_{2} ( {P}_{C , {D}_{2}} ⊙ {w}_{1 to 2} ) \qquad [ Equation 5 ]$$

$$α , β = softmax ( {α}_{pre} ‖ {β}_{pre} ) \qquad [ Equation 6 ]$$

In [Equation 4] to [Equation 6], αₚᵣₑ denotes the first output information, βₚᵣₑ denotes the second output information, E denotes efficacy, DNN₂ denotes a deep-learning neural network included in the sixth network 222, a subscript dᵢ denotes the concentration of the i-th drug, ⊙ denotes an element-wise product, and [a∥b] denotes the concatenation of vectors a and b, respectively. For example, E_{C,D}_{₁}_{,d}_{₁} denotes efficacy for which the cell line C, the first drug D₁, and the first concentration d₁ of the first drug are used as input information to the first neural network model 210.

The seventh network 223 includes a deep-learning neural network (DNN) structure. Although the architecture of the DNN structures of the sixth network 222 and the seventh network 223 may be the same, their training parameters may be configured differently.

The seventh network 223 receives, as input information, the concatenation of the product of the first efficacy information ES1 and the first pathway attention PA1, and the product of the second efficacy information ES2 and the second pathway attention PA2, and outputs synergistic efficacy γ. The synergistic efficacy γ is input to the efficacy parameter output unit 225.

The computation of the synergistic efficacy may be represented as in [Equation 7].

$$γ = {DNN}_{3} ( [ {E}_{C , {D}_{1} , {d}_{1}} * {P}_{C , {D}_{1}} ‖ {E}_{C , {D}_{2} , {d}_{2}} * {P}_{C , {D}_{2}} ] ) \qquad [ Equation 7 ]$$

Here, γ denotes synergistic efficacy, DNN₃ denotes a deep-learning neural network included in the seventh network 223, and [a∥b] denotes the concatenation of vectors a and b.

The efficacy parameter output unit 225 outputs combination efficacy information EC by summing the product of the first efficacy information ES1 and the first efficacy ratio α, the product of the second efficacy information ES2 and the second efficacy ratio β, and the synergistic efficacy γ.

The combination efficacy information EC may be represented as in [Equation 8].

$${E}_{C , {D}_{2} , {d}_{2} , {d}_{2}} = α {E}_{C , {D}_{1} , {d}_{1}} + β {E}_{C , {D}_{2} , {d}_{2}} + γ \qquad [ Equation 8 ]$$

Here, the combination efficacy information E_{C,D}_{₁}_{,D}_{₂}_{,d}_{₁}_{,d}_{₂} denotes combination efficacy information when the cell line C, the first drug D₁, the second drug D₂, the first concentration d₁ of the first drug, and the second concentration d₂ of the second drug are used as input information.

Meanwhile, cancer cell viability V and efficacy E are related by [Equation 9].

$$V = 1 - E \qquad [ Equation 9 ]$$

The second neural network model 220 is trained by the third dataset described above. Since the third dataset includes response information on combination anticancer drugs, the second neural network model 220 may effectively learn combination efficacy through the third dataset.

In the training process of the second neural network model 220, parameters of the networks included in the second neural network model 220 are adjusted, but the parameters included in the first neural network model 210 are not adjusted and remain frozen.

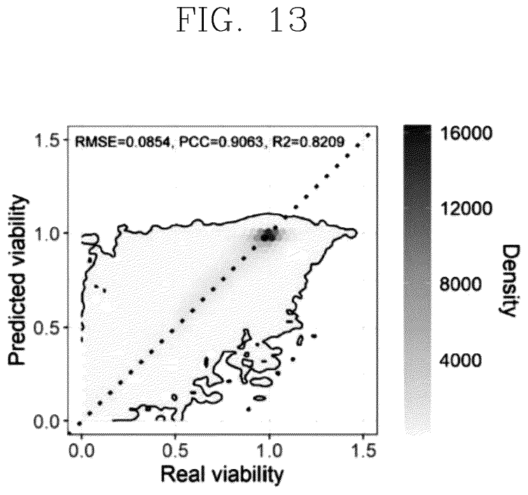

FIG. 13 illustrates the performance of the second neural network model 220.

The performance of the trained second neural network model 220 is visualized as a graph in which the viability of the validation data (Real viability, x-axis), the viability of the predicted data (Predicted viability, y-axis), and the density of data (represented by color intensity) are plotted.

On the top of the graph, the Root Mean Square Error (RMSE), Pearson Correlation Coefficient (PCC), and R-squared (R2) are displayed respectively.

The RMSE, PCC, and R2 of the trained second neural network model 220 are 0.0854, 0.9063, and 0.8209, respectively.

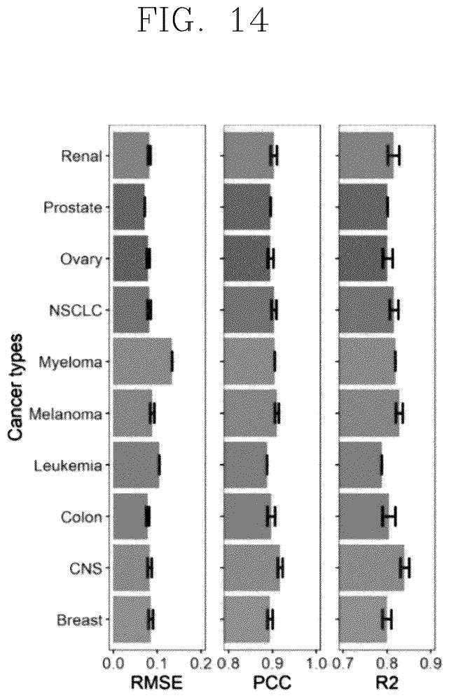

FIG. 14 illustrates the performance of the system 10 for predicting efficacy of a combination anticancer drug depending on cancer type.

It can be seen that the system 10 for predicting efficacy of a combination anticancer drug according to an embodiment of the present invention shows high performance regardless of cancer type.

The communication unit 300 may transmit and receive information with an external device 400 under control of the processor 100.

The communication unit 300 may communicate using at least one method among wired/wireless LAN, Wi-Fi (wireless fidelity), Bluetooth, Zigbee, infrared communication (IrDA, infrared Data Association), near field communication (NFC), wireless broadband internet (WiBro), shared wireless access protocol (SQAP), and RF communication, but the communication method is not necessarily limited to the above-described embodiment.

The external device 400 may refer to any type of device that transmits and receives information with the system 10.

For example, the external device 400 may transmit information requesting inference of combination efficacy information, along with provision of cancer cell information (corresponding to cell line information), the first drug and first concentration, and the second drug and second concentration to the system 10.

For example, the external device 400 may be an external server that provides training data used for training.

For example, the external device 400 may transmit a control command to the system 10 to control partial operations of the system 10.

For example, the external device 400 may transmit setting conditions to the system 10 and receive drug concentration suggestion information as a response.

For example, the external device 400 may be any one of a server, a smartphone, a tablet PC, a desktop PC, or a notebook, but is not limited to these examples.

Hereinafter, a method for predicting efficacy of a combination anticancer drug will be described in detail, by way of example, as being performed by the system 10 for predicting efficacy of a combination anticancer drug.

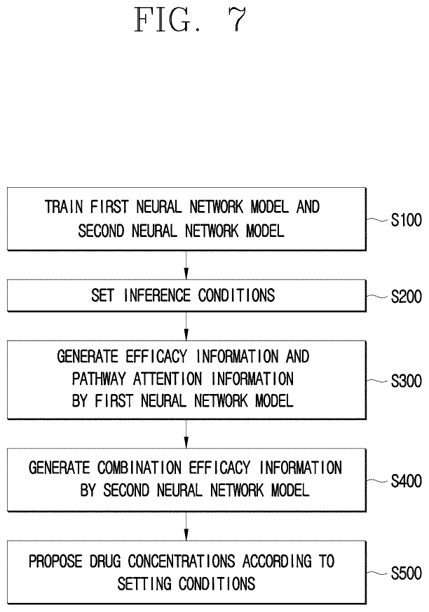

FIG. 7 illustrates a method for predicting efficacy of a combination anticancer drug according to an embodiment of the present invention.

With reference to FIG. 7, in step S100, the system 10 performs training for the first neural network model 210 and the second neural network model 220.

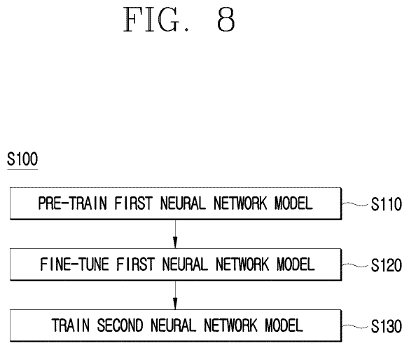

FIG. 8 illustrates step S100 in detail.

With reference to FIG. 8, in step S110, the system 10 performs pre-training for the first neural network model 210.

The pre-training may be performed by setting the first dataset described above as training data.

In step S120, the system 10 performs fine-tuning for the first neural network model 210.

The fine-tuning may be performed by setting the second dataset described above as training data.

After the fine-tuning, parameters of the first neural network model 210 may be frozen.

In step S130, the system 10 performs training for the second neural network model 220.

The training for the second neural network model 220 may be performed by setting the second dataset described above as training data.

With reference back to FIG. 7, in step S200, the system 10 sets inference conditions.

The inference conditions refer to conditions regarding cancer cell information (target protein information), the first drug to be included in the combination anticancer drug, the first concentration of the first drug, the second drug, and the concentration of the second drug. The concentration may also be input by vectorizing the condition of the setting range. In this case, the combination efficacy information may be data distributed with the first concentration and second concentration as variables.

Setting inference conditions refers to determining the inference conditions, and may be set through an input interface to be provided in the system 10 or through transmission of information by the external device 400.

In step S300, the system 10 generates efficacy information and pathway attention information by the first neural network model 210.

Step S300 is performed by using the first drug, first concentration, and target cancer cell information as input information, and is performed simultaneously or sequentially by using the second drug, second concentration, and target cancer cell information as input information.

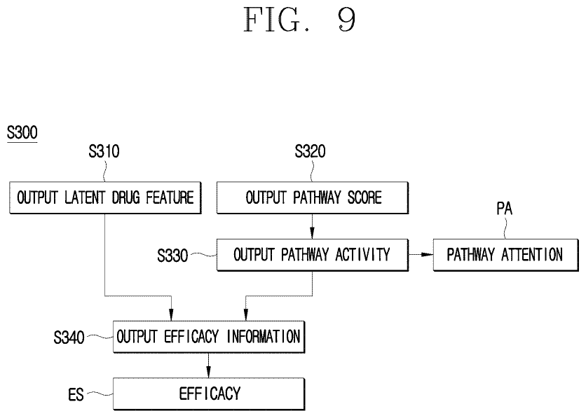

FIG. 9 illustrates step S300 in detail.

With reference to FIG. 9, in step S310, the first network 211 outputs latent drug feature information from drug information input in the form of a Morgan fingerprint.

In step S320, the second network 212 outputs a pathway score from cell line information of a cancer cell and drug information input in the form of a Morgan fingerprint. The pathway score is input to the third network 213.

In step S330, the third network 213 outputs a pathway activity from the pathway score and drug information input in the form of a Morgan fingerprint. The pathway activity is input to the fourth network 214.

In the process of performing step S330, pathway attention PA is generated, and the pathway attention PA information is used in step S400.

In step S340, the fourth network 214 outputs efficacy information ES by using the concatenated latent drug feature and pathway activity as input. The efficacy information may be information expressed as E=1−V (V is the viability of the target cancer cell for a specific drug and concentration).

Since step S300 is performed for two drugs, the generated results of step S300 used in performing step S400 may include first pathway attention PA1, second pathway attention PA2, first efficacy information ES1, and second efficacy information ES2.

In step S400, the system 10 generates combination efficacy information by the second neural network model 220.

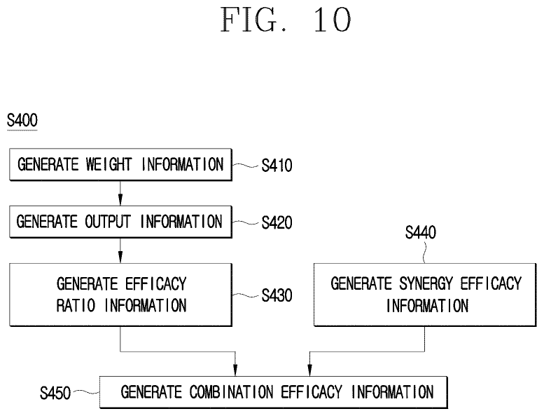

FIG. 10 illustrates step S400 in detail.

With reference to FIG. 10, in step S410, the fifth network 221 generates the first weight w12 from the first pathway attention PA1 and the second weight w21 from the second pathway attention PA2, respectively.

In step S420, the sixth network 222 generates first output information by using the multiplied first weight w12 and second pathway attention PA2 as input, and generates second output information by using the second weight w21 and first pathway attention PA1 as input, respectively.

The first output information is multiplied with the first efficacy information ES1, and the second output information is multiplied with the second efficacy information ES2, and the results of the respective multiplications are input to the ratio operation unit 224 as concatenated efficacy concatenation information.

In step S430, the ratio operation unit 224 generates the first efficacy ratio α and the second efficacy ratio β, respectively, using the concatenated efficacy concatenation information as input.

In step S440, the seventh network 223 receives, as input information, the concatenation of the product of the first efficacy information ES1 and the first pathway attention PA1, and the product of the second efficacy information ES2 and the second pathway attention PA2, and generates synergistic efficacy γ.

In step S450, the efficacy parameter output unit 225 generates combination efficacy information EC by summing the product of the first efficacy information ES1 and the first efficacy ratio α, the product of the second efficacy information ES2 and the second efficacy ratio β, and the synergistic efficacy γ.

The combination efficacy information EC may be output as data regarding efficacy for a target cancer cell, with the first concentration of the first drug and the second concentration of the second drug as respective variables.

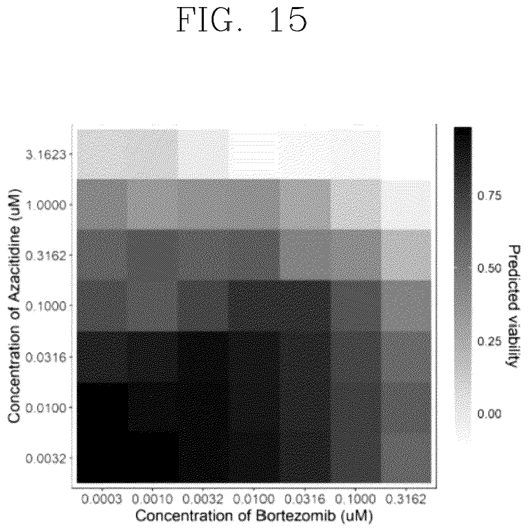

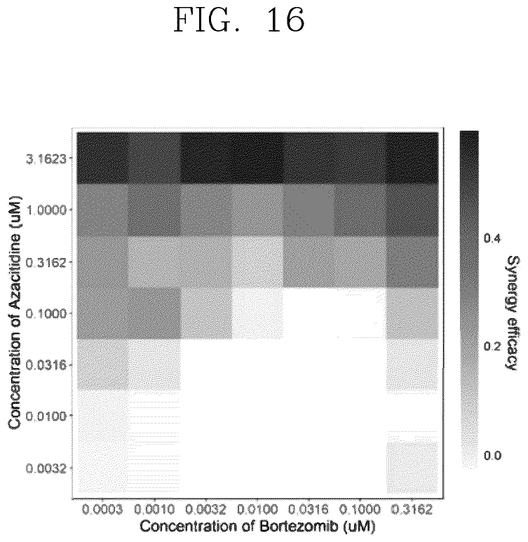

FIGS. 15 and 16 illustrate viability and synergistic effect depending on the concentration of component drugs of the combination anticancer drug.

The target cancer cell is RPMI-8226, which is a multiple myeloma cancer cell, and the first drug is set to Bortezomib, and the second drug is set to Azacitidine.

With regard to the action of the second drug, the second drug shows a tendency to decrease viability and increase synergistic efficacy as its concentration increases. In contrast, the first drug did not show a tendency of a significant increase in viability and synergistic efficacy compared to increasing concentration. For example, administering 0.01 μM of the first drug showed a higher synergistic effect rather than administering 0.3182 μM.

With reference to the efficacy and synergistic effect distributions illustrated in FIGS. 15 and 16, it can be seen that to achieve a certain level of combination efficacy, the concentration of the second drug needs to be relatively high, but the first drug may contribute to synergistic effect and combination efficacy enhancement even in a relatively small amount.

In the anticancer drug field, since there is a trade-off relationship between efficacy and side effects depending on the drug concentration, the present invention has the effect of being able to obtain concentration information that achieves desired efficacy while minimizing side effects of the combination anticancer drug.

In step S500, the system 10 proposes drug concentrations according to the setting conditions.

Here, the setting conditions may be concentration conditions of at least one of the first drug or the second drug. For example, the concentration condition of the first drug may be equal to or less than the set value. The setting condition may further include a condition regarding the combination efficacy. For example, the setting condition may be that the first drug is equal to or less than the set concentration and the combination efficacy is equal to or greater than the set value.

The system 10 proposes a concentration of at least one of the first drug or the second drug corresponding to the setting condition. For example, the system 10, under a condition in which the first drug is equal to or less than a set concentration A and the combination efficacy is equal to or greater than B, may specify each of the concentration of the first drug and the concentration of the second drug, or set a range thereof for provision. Of course, the corresponding drug concentration is for targeting a specific cancer cell.

Further, the system 10 for predicting efficacy of a combination anticancer drug according to the present invention may be implemented through a computing device to be described below, and may perform data processing related to the method for predicting efficacy of a combination anticancer drug as described above.

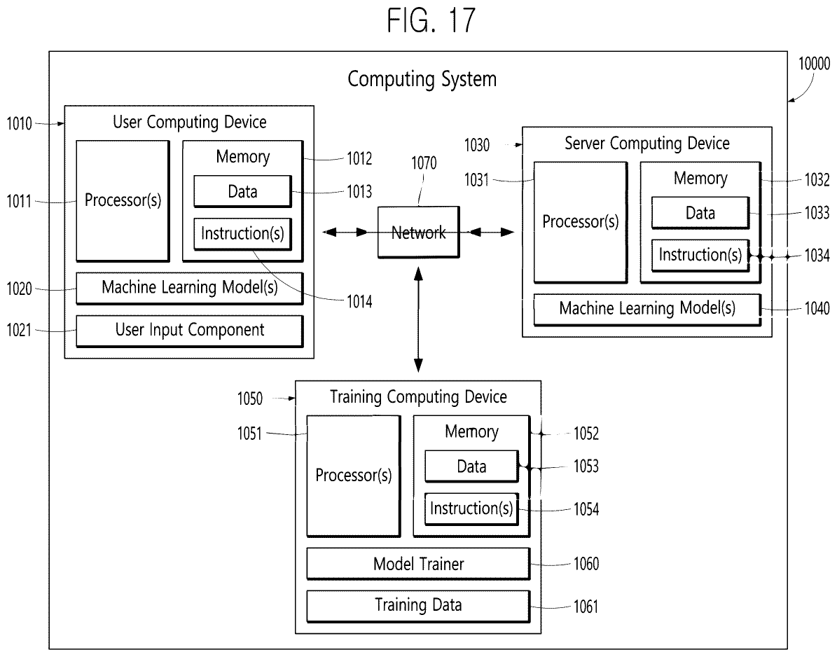

FIG. 17 illustrates an example block diagram of a computing system in which the present invention may be implemented.

Referring to FIG. 17, a computing system (10000) for performing a method for estimating efficacy of combination anticancer drug according to an embodiment of the present invention may include at least one computing device. In this case, the at least one computing device may be a single-processor or multi-processor computing apparatus.

The components of the at least one computing device of the present invention may include one or more processors, memory, other hardware, and various system components connected (e.g., communicatively, physically, or electrically connected) via a system bus (not shown) that enables data to be transmitted and received among them. The components of the at least one computing device are not limited thereto and may vary widely.

Meanwhile, the at least one computing device included in the computing system (10000) that performs a method for estimating efficacy of combination anticancer drug may be communicatively connected via a network (1070). For example, the at least one computing device included in the computing system (10000) may be clustered or may be part of a local area network (LAN). Additionally, the at least one computing device may be part of a wide area network (WAN) or connected via at least one of a client-server network or a peer-to-peer network in a cloud environment.

Meanwhile, when the at least one computing device is used in at least one environment among a network environment and a cloud computing environment, the at least one computing device may be connected to at least one of a public network and a private network through a network interface or adapter. In one embodiment, other communication connection devices, such as a modem, may be used to establish communication over the network. The modem may be at least one of an internal modem and an external modem, and may be connected to the system bus through a network interface or a specific mechanism. A wireless network component comprising an interface and an antenna may be coupled to the network through devices such as access points or peer computers. In the present invention, the method by which the at least one computing device is communicatively connected via the network (1070) is not limited thereto and may be implemented by means other than the examples described above.

Furthermore, other computer-type devices and/or systems not illustrated in FIG. 17 may technically interact with the at least one computing device or other systems through one or more connections to the network (1070) via a network interface. Here, the network interface may include network interface equipment such as a physical Network Interface Controller (NIC) or a Virtual Interface (VIF).

The network (1070) of the present invention may include various types of networks such as the Internet, Wireless LAN (WLAN), Wireless Fidelity (Wi-Fi), Digital Living Network Alliance (DLNA), Wireless Broadband (WiBro), Worldwide Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A), 5th Generation Mobile Telecommunication (5G), Bluetooth™, Radio Frequency Identification (RFID), Infrared Data Association (IrDA), Ultra-Wideband (UWB), ZigBee, Near Field Communication (NFC), Wi-Fi Direct, Wireless Universal Serial Bus (Wireless USB), and the like. In the present invention, data transmission may be performed based on standard communication protocols such as TCP/IP, HTTP, SSL, and others.

The computing system (10000) for performing a problem-solving a method for estimating efficacy of combination anticancer drug according to the present invention may include at least one of a user computing device (1010), a training computing device (1050), and a server computing device (1030).

The user computing device (1010) according to the present invention may be understood as a computing device including at least one processor (1011) and memory (1012) for performing the method for estimating efficacy of combination anticancer drug. For example, the user computing device (1010) may include at least one computing device selected from among a smart phone, smart TV, laptop computer, desktop computer, digital broadcasting terminal, personal digital assistant (PDA), portable multimedia player (PMP), navigation device, slate PC, tablet PC, ultrabook, and wearable device (e.g., smartwatch, smart glass, and head-mounted display (HMD)).

The at least one processor (1011) constituting the user computing device (1010) may include one or more general-purpose processors and/or one or more special-purpose processors. For example, the at least one processor (1011) of the user computing device (1010) may include at least one or a combination of electrically connected processors selected from the group consisting of: a Central Processing Unit (CPU), a Graphics Processing Unit (GPU), a Tensor Processing Unit (TPU), a Neural Processing Unit (NPU), an Arithmetic Logic Unit (ALU), a Floating Point Unit (FPU), an Application-Specific Integrated Circuit (ASIC), a digital signal processing device (DSPD), a programmable logic device (PLD), a Field Programmable Gate Array (FPGA), a controller, a microcontroller, a microprocessor, and other electrical units for performing specific functions.

Furthermore, the at least one processor (1011) may be configured to execute computer-readable instructions stored in the memory (1012) and/or other commands described in the present specification.

The memory (1012) constituting the user computing device (1010) according to the present invention may include volatile memory, non-volatile memory, fixed media, removable media, magnetic media, optical media, semiconductor media, and/or other types of physically durable storage media.

For example, the memory (1012) may include one or more non-transitory/transitory computer-readable storage media, or combinations thereof, such as Random Access Memory (RAM), Read Only Memory (ROM), Hard Disk Drive (HDD), Solid State Disk (SSD), Silicon Disk Drive (SDD), Electrically Erasable Programmable Read-Only Memory (EEPROM), Erasable Programmable Read-Only Memory (EPROM), flash memory devices, and magnetic disks. It may also include web storage of a server that performs the memory storage function over the Internet.

The memory (1012) may store data and instructions necessary for the at least one processor (1011) to perform operations of an application for implementing a method for estimating efficacy of combination anticancer drug.

The user computing device (1010) may include one or more user input components (1021) configured to detect user input. For example, the user input component (1021) may also be referred to as a user interface module. The user input component (1021) may include devices such as a touchscreen, computer mouse, keyboard, keypad, touchpad, trackball, joystick, voice recognition module, or other similar devices. However, the present invention does not limit the types of the user input component (1021).

In this context, the user input component (1021) in the present invention is not necessarily limited to a hardware means but may be understood as a channel through which input is received from a user.

Meanwhile, the “user” in the present invention may also refer to an automated agent, script, playback software, or the like that operates on behalf of one or more human users.

A user may interact with the computing system (10000), which includes at least one computing device, through the user input component (1021) using inputted text, touch, voice, motion, computer vision, gesture, and/or other forms of input/output. For example, the user input component (1021) may include one or more user interface (UI) modalities such as a Command Line Interface (CLI), Graphical User Interface (GUI), Natural User Interface (NUI), voice command interface, and/or other UI representations.

One or more Application Programming Interface (API) calls may be made between the user input component (1021) and the user computing device (1010), based on user input received through a user interface and/or from a network.

Herein, the phrase “based on” may be interpreted to include instances where a particular configuration is used as a foundation, modified from, derived from, influenced by, dependent on, or otherwise originating from such configuration.

In some embodiments, the API call may be configured for a specific API and may be interpreted as, or converted into, an API call configured for a different API. In this context, the API may refer to a defined interface or connection between computers or between computer programs.

In one embodiment, the user computing device (1010) may store one or more machine learning models (1020). For example, the user computing device (1010) may include various machine learning models, such as multiple neural networks (e.g., deep neural networks) for performing a method for estimating efficacy of combination anticancer drug using drug information, or other types of machine learning models including nonlinear models and/or linear models or may be configured as a combination thereof.

According to an embodiment of the present invention, the user computing device (1010) may perform a method for estimating efficacy of combination anticancer drug by using a local and/or external machine learning model (1020). Alternatively, the user computing device (1010) may perform the method for estimating efficacy of combination anticancer drug by using a machine learning model (1040) provided by a server.

According to another embodiment of the present invention, a server computing device (1030) communicating with the user computing device (1010) may provide combination efficacy information to the user computing device (1010) via an application and/or a web interface, in response to a user request received through the user computing device (1010).

According to yet another embodiment of the present invention, at least a portion of the user computing device (1010) and the server computing device (1030) may be cooperatively operated to perform a method for estimating efficacy of combination anticancer drug, thereby providing combination efficacy information to the user.

According to various embodiments of the present invention, the user computing device (1010) and/or the server computing device (1030) may train the machine learning models (1020, 1040) used in the method for estimating efficacy of combination anticancer drug through interaction with a training computing device (1050) that is communicatively connected via the network (1070).

In this case, the training computing device (1050) may be a computing system separate from the server computing device (1030). Alternatively, in some embodiments, the training computing device (1050) may be a part of the server computing device (1030) or a part of the user computing device (1010).

Meanwhile, the server computing device (1030) may include at least one processor (1031) and memory (1032). Here, the processor (1031) may include at least one or a combination of electrically connected processors selected from among: a Central Processing Unit (CPU), Graphics Processing Unit (GPU), Tensor Processing Unit (TPU), Neural Processing Unit (NPU), Application-Specific Integrated Circuit (ASIC), Arithmetic Logic Unit (ALU), Floating Point Unit (FPU), digital signal processing devices (DSPDs), programmable logic devices (PLDs), Field Programmable Gate Arrays (FPGAs), controllers, microcontrollers, microprocessors, and/or other electrical units for performing specific functions. For example, the at least one processor (1031) may include circuits and transistors configured to execute instructions from the memory (1032).

The memory (1032) constituting the server computing device (1030) according to the present invention may include volatile memory, non-volatile memory, fixed media, removable media, magnetic media, optical media, semiconductor media, and/or other types of physically durable storage media.

For example, the memory (1032) may include one or more transitory/non-transitory computer-readable storage media, or combinations thereof, such as Random Access Memory (RAM), Read Only Memory (ROM), Hard Disk Drive (HDD), Solid State Disk (SSD), Silicon Disk Drive (SDD), Electrically Erasable Programmable Read-Only Memory (EEPROM), Erasable Programmable Read-Only Memory (EPROM), flash memory devices, and magnetic disks. It may also include web storage of a server that performs memory storage functions over the Internet.

Additionally, the server computing device (1030) may further include a data store. For example, the data store may be configured as at least one of a relational database, a NoSQL database, a data warehouse, and a local file system.

The memory (1032) constituting the server computing device (1030) according to the present invention may store data and instructions necessary for the at least one processor (1031) to perform operations of an application for implementing a method for estimating efficacy of combination anticancer drug.

In one embodiment, the server computing device (1030) may be configured as a single device or as a plurality of computing devices, which may be configured to operate according to a sequential or parallel computing architecture. Additionally, the system may be implemented as a distributed processing system comprising multiple devices connected over a network.

Meanwhile, the training computing device (1050) may include at least one processor (1051) and memory (1052). A model trainer (1060), as a logical component that performs training of at least one machine learning model (1020, 1040), may be implemented in the form of hardware, firmware, or software.

For example, the model trainer (1060) may load training data (1061) stored in a storage device into the memory (1052), and then be executed by the processor (1051). The model trainer (1060) may be configured to perform one or more operations-such as model training, model reconstruction, model validation, and model testing-on at least one machine learning model.

The machine learning model according to the present invention may include at least one of the following: a statistical model, an algorithm, a neural network (NN), a convolutional neural network (CNN), a generative neural network (GNN), a Word2Vec model, a Bag of Words model, a Term Frequency-Inverse Document Frequency (TF-IDF) model, a Generative Pre-trained Transformer (GPT) model (or other autoregressive models), a Proximal Policy Optimization (PPO) model, a nearest neighbor model (e.g., k-nearest neighbor model), a linear regression model, a k-means clustering model, a Q-learning model, a Temporal Difference (TD) model, a Deep Adversarial Network model, and any other type of model described in the present specification.

Specifically, the model trainer (1060) may perform operations for training a machine learning model, and the operations may include at least one of adding, removing, and modifying model parameters. In this case, the training of the machine learning model may be at least one of supervised learning, semi-supervised learning, and unsupervised learning.

In one embodiment, training of the machine learning model may include a step of repeatedly inputting the training data (1061) based on epochs, and iteratively performing the machine learning model training process configured in this manner. Here, an epoch may refer to a unit representing one complete forward and backward pass of the entire training data (1061) set.

In some implementations, different learning methods (e.g., supervised learning, semi-supervised learning, and unsupervised learning) may be applied at different epochs.

The training data (1061) of the present invention may include input data and/or data previously output from at least one machine learning model (e.g., recursive learning feedback).

The parameters of the at least one machine learning model may include at least one of a seed value, model nodes, model layers, algorithms, functions, connections between different machine learning models, connections between parameters, constraints of the machine learning model, and other digital components that influence the output of the machine learning model.

In this case, a model connection between different machine learning models may include or represent relationships between model parameters and/or between models, which may be dependent, interdependent, hierarchical, and/or static or dynamic.

The combination and configuration of the model parameters described herein may be too complex to be maintained or utilized by human cognitive capabilities.

The present invention does not limit the parameters of machine learning models to those described in the embodiments, and a single machine learning model may include a plurality of model parameters.

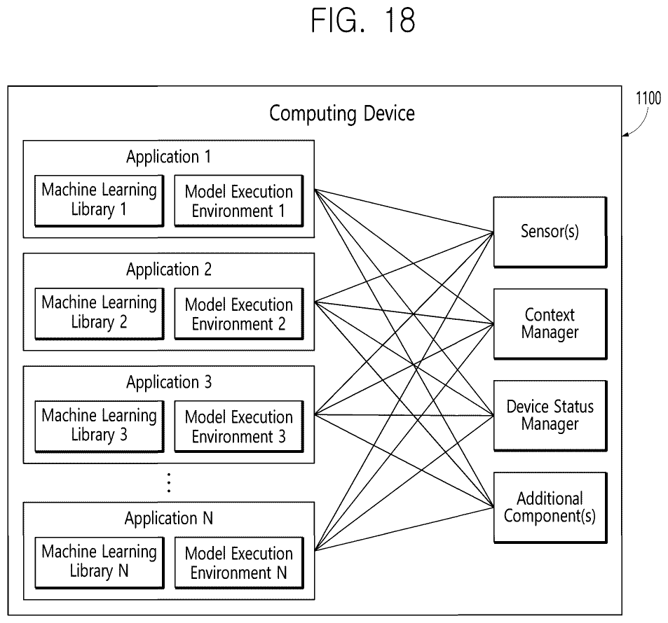

Meanwhile, FIG. 18 illustrates an example block diagram of a computing device (1100), which may be included in the user computing device (1010), the server computing device (1030), or the training computing device (1050), as one embodiment of the computing system (10000) in which the present invention may be implemented.

As shown in FIG. 18, the computing device (1100) may include at least one application (e.g., Application 1 to Application N), and each of the at least one application may include a machine learning library and a model execution environment for performing a method for estimating efficacy of combination anticancer drug using machine learning.

Each of the at least one application included in the computing device (1100) may communicate via an Application Programming Interface (API) with one or more components within the computing device (1100), such as sensors, a context manager, a device state manager, or additional components.

In one embodiment, the at least one application may interface with device components by, for example, receiving sensor data or state data via a public or dedicated API, or transmitting prediction results to an output device.

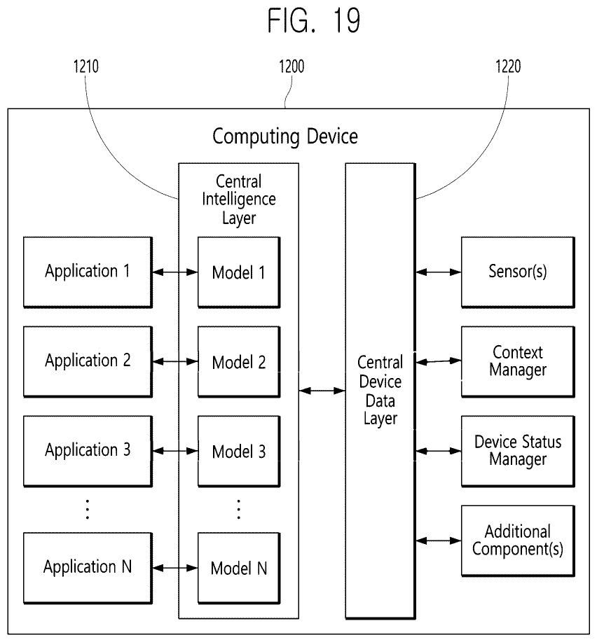

Meanwhile, FIG. 19 illustrates an example block diagram of a computing device (1200), which is one component of the computing system (10000) performing the method for estimating efficacy of combination anticancer drug according to an embodiment of the present invention, from another perspective.

The computing device (1200) according to the present invention may include at least one application (e.g., Application 1 to Application N), and each of the at least one application may communicate with a central intelligence layer (1210). Each application may interact with a shared model within the central intelligence layer (1210) via an API (e.g., a common API).

The central intelligence layer (1210) may include one or more machine learning models and may either share them among multiple applications or provide them independently to each application. In one embodiment, the central intelligence layer (1210) may be integrated as part of the operating system or implemented as a separate logical layer.

Additionally, the central intelligence layer (1210) may communicate with a central device data layer (1220). The central device data layer (1220) may integratively store drug information and the like stored within the computing device (1200) and provide it as input data required for implementing a method for estimating efficacy of combination anticancer drug. Each device component (e.g., sensors, state managers, etc.) may communicate with the central device data layer (1220) via a private API or the like.

The technology described in the present specification may be implemented using a single computing device or multiple computing devices. A machine learning model for implementing a method for estimating efficacy of combination anticancer drug may be executed sequentially or in parallel on a single component or across multiple distributed components. The data store, machine learning models, and applications may be distributed and operated locally or over a network, and these components may be flexibly applied to various system architectures.

The above has described the implementation of the system 10 for predicting efficacy of a combination anticancer drug of the present invention as a computing system, but the present invention is not limited thereto. For example, the functionality of the neural network and/or computing device may be distributed among a plurality of computing clusters.

Meanwhile, the present invention described above may be executed by one or more processes on a computer and implemented as a program that may be stored on a computer-readable medium (or recording medium).

Further, the present invention described above may be implemented as computer-readable code or instructions on a medium in which a program is recorded. That is, the present invention may be provided in the form of a program.

Meanwhile, the computer-readable medium includes all kinds of recording devices for storing data readable by a computer system. Examples of computer-readable media include hard disk drives (HDDs), solid state disks (SSDs), silicon disk drives (SDDs), ROMs, RAMs, CD-ROMs, magnetic tapes, floppy discs, and optical data storage devices.

Further, the computer-readable medium may be a server or cloud storage that includes storage and that the electronic device is accessible through communication. In this case, the computer may download the program according to the present invention from the server or cloud storage, through wired or wireless communication.

Further, in the present invention, the computer described above is an electronic device equipped with a processor, that is, a central processing unit (CPU), and is not particularly limited to any type.

Meanwhile, it should be appreciated that the detailed description is interpreted as being illustrative in every sense, not restrictive. The scope of the present invention should be determined on the basis of the reasonable interpretation of the appended claims, and all of the modifications within the equivalent scope of the present invention belong to the scope of the present invention.

The terminology used herein is used for the purpose of describing particular embodiments only and is not intended to limit the present invention. The terms “comprises,” “comprising,” “includes,” “including,” “containing,” “has,” “having” or other variations thereof are inclusive and therefore specify the presence of stated features, integers, steps, operations, elements, and/or components, but do not preclude the presence or addition of one or more other features, integers, steps, operations, elements, components, and/or groups thereof.

### DESCRIPTION OF REFERENCE NUMERALS

10: System
100: Processor
200: Memory
210: First neural network model
211: First network
212: Second network
213: Third network
214: Fourth network
220: Second neural network model
221: Fifth network
222: Sixth network
223: Seventh network
224: Ratio operation unit
225: Efficacy parameter output unit
300: Communication unit
400: External device

What is claimed is:

1. A system for predicting efficacy of a combination anticancer drug, the system comprising:
a processor configured to compute efficacy of a combination anticancer drug based on first drug information and second drug information using a neural network model; and
a memory,
wherein the memory comprises:
a first neural network model configured to generate first efficacy-related information from the first drug information and target cancer cell information, and to generate second efficacy-related information from the second drug information and the target cancer cell information; and
a second neural network model configured to generate combination efficacy information from the first efficacy-related information, the second efficacy-related information, and the target cancer cell information, and
wherein the first efficacy-related information and the second efficacy-related information are respectively generated based on a first concentration of the first drug and a second concentration of the second drug.

2. The system of claim 1, wherein the first efficacy-related information includes first pathway attention information, and the second efficacy-related information includes second pathway attention information.

3. The system of claim 1, wherein the second neural network model includes at least one deep-learning neural network (DNN) for computing a first efficacy ratio in which the first drug contributes to the combination efficacy, and a second efficacy ratio in which the second drug contributes to the combination efficacy.

4. The system of claim 3, wherein the second neural network model further comprises a synergy computation network for computing synergistic efficacy in which a synergistic effect of the first drug and the second drug contributes to the combination efficacy.

5. The system of claim 1, wherein the first neural network model is pre-trained with a first dataset determined from a first database which includes more response information of a target cancer cell to a single drug than a second database, and is then fine-tuned with a second dataset including single drug information, target cancer cell information, and single drug response information of a second database, in which response information of the target cancer cell to both the single drug and a combination drug formed by combining the single drug is present.

6. The system of claim 5, wherein the second neural network model is trained with a third dataset including combination drug information, target cancer cell information, and combination drug response information from the second database.

7. A method for predicting efficacy of a combination anticancer drug, executed by at least one processor of a computing device, the method comprising:
determining first drug information, second drug information, and target cancer cell information;
generating first efficacy-related information from the first drug information and the target cancer cell information;
generating second efficacy-related information from the second drug information and the target cancer cell information; and
generating combination efficacy information from the first efficacy-related information, the second efficacy-related information, and the target cancer cell information,
wherein the first efficacy-related information and the second efficacy-related information are respectively generated based on a first concentration of the first drug and a second concentration of the second drug.

8. The method of claim 7,
wherein the first efficacy-related information includes first pathway attention information, and the second efficacy-related information includes second pathway attention information.

9. The method of claim 7,
wherein generating the combination efficacy information comprises using a second neural network model including at least one deep-learning neural network (DNN) for computing a first efficacy ratio in which the first drug contributes to the combination efficacy, and a second efficacy ratio in which the second drug contributes to the combination efficacy.

10. The method of claim 9,
wherein the second neural network model further comprises a synergy computation network for computing synergistic efficacy in which a synergistic effect of the first drug and the second drug contributes to the combination efficacy.

11. The method of claim 7,
wherein generating the first efficacy-related information comprises using a first neural network model that is pre-trained with a first dataset determined from a first database which includes more response information of a target cancer cell to a single drug than a second database, and then fine-tuned with a second dataset including single drug information, target cancer cell information, and single drug response information of a second database, in which response information of the target cancer cell to both the single drug and a combination drug formed by combining the single drug is present.

12. The method of claim 11,
wherein generating the combination efficacy information comprises using the second neural network model trained with a third dataset including combination drug information, target cancer cell information, and combination drug response information from the second database.

13. A program stored in a non-transitory computer-readable storage medium, executed by one or more processes in an electronic device, wherein the program includes instructions to perform:
determining first drug information, second drug information, and target cancer cell information;
generating first efficacy-related information from the first drug information and the target cancer cell information;
generating second efficacy-related information from the second drug information and the target cancer cell information; and
generating combination efficacy information from the first efficacy-related information, the second efficacy-related information, and the target cancer cell information,
wherein the first efficacy-related information and the second efficacy-related information are respectively generated based on a first concentration of the first drug and a second concentration of the second drug.

14. The non-transitory computer-readable storage medium of claim 13,
wherein the instructions, when executed by one or more processors, cause the one or more processors to generate the first efficacy-related information including first pathway attention information, and the second efficacy-related information including second pathway attention information.

15. The non-transitory computer-readable storage medium of claim 13,
wherein the instructions, when executed by one or more processors, cause the one or more processors to generate the combination efficacy information using a second neural network model including at least one deep-learning neural network (DNN) for computing a first efficacy ratio in which the first drug contributes to the combination efficacy, and a second efficacy ratio in which the second drug contributes to the combination efficacy.

16. The non-transitory computer-readable storage medium of claim 15,
wherein the instructions, when executed by one or more processors, cause the one or more processors to utilize the second neural network model further comprising a synergy computation network for computing synergistic efficacy in which a synergistic effect of the first drug and the second drug contributes to the combination efficacy.

17. The non-transitory computer-readable storage medium of claim 13,
wherein the instructions, when executed by one or more processors, cause the one or more processors to generate the first efficacy-related information using a first neural network model that is pre-trained with a first dataset determined from a first database which includes more response information of a target cancer cell to a single drug than a second database, and then fine-tuned with a second dataset including single drug information, target cancer cell information, and single drug response information of a second database, in which response information of the target cancer cell to both the single drug and a combination drug formed by combining the single drug is present.

18. The non-transitory computer-readable storage medium of claim 17,
wherein the instructions, when executed by one or more processors, cause the one or more processors to generate the combination efficacy information using the second neural network model trained with a third dataset including combination drug information, target cancer cell information, and combination drug response information from the second database.
